(19) United States
(12) Patent Application Publication
(10) Pub. No.: US 2003/0095902 A1
(43) Pub. Date: May 22, 2003

(54) Photocatalytic electric fan

(76) Inventors: Yuan Huan Lee, Hsin Chu Hsien (TW); Wen Hu Cheng, Hsin Chu Hsien (TW); Chu Chun Huang, Hsin Chu Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/988,089

(22) Filed: Nov. 19, 2001

(51) Int. Cl. A62B 7/08; A61L 9/00

(52) U.S. Cl. 422/306; 422/121; 422/124; 422/307

(57) ABSTRACT

A photocatalytic electric fan comprises a main body having a receiving tank therein. A motor is fixed in the receiving tank. A rotation shaft of the motor is connected with a fan blade. A lamp-fixing element is joined at an opening of the main body near the fan blade. A photocatalytic lamp is positioned on the lamp-fixing element. At least a photocatalytic lamp tube is disposed on the lamp. Each photocatalytic lamp tube is connected to an electric connection seat, a stabilizer, a starter, a switch, and a power source. A face net is joined at the other opening of the main body. The face net is rotatable. The photocatalytic lamp tube is a UV lamp tube with a glass-fiber-cloth sleeved outside. The glass-fiber-cloth has a photocatalytic coating formed of titanium dioxide.

### FIELD OF THE INVENTION

The present invention relates to a photocatalytic electric fan and, more particularly, to a structure for cleaning air passing through an electric fan.

### BACKGROUND OF THE INVENTION

For people inhabit the tropics and subtropics, in addition to air conditioners, electric fans are mostly used to endure hot and wet weathers. As shown in FIG. 1, a box type electric fan comprises a seat body 11, a motor 12, a fan blade 13, a back net 14, and a face net 15. The seat body 11 is composed of a front shell 16 and a rear shell 17 locked together with a long screw 10. A bottom seat 18 is pivotally joined on the seat body 11 so that the seat body 11 can be tuned over back and forth. The face net 15 is rotatable. The back net 14 is integrally formed, and is locked on the rear shell 17 with a plurality of screws 19. A switch button and several rotation speed selection buttons are disposed on the seat body 11. For another type of seat body, two ends thereof can be pivotally joined on a base (not shown) according to necessity to be capable of turning over.

Although electric fans are widely used to generate wind for fanning people, it has only this function. No matter what kind of air sucked in from the rear side of an electric fan, the air is blown forwards. No further processing can be performed to the sucked-in air. In other words, the electric fan is only a tool for circulation of air, and has no other functions. The present invention aims to resolve the above problem in the prior art.

### SUMMARY OF THE INVENTION

The object of the present invention is to provide a photocatalytic electric fan, wherein a specially designed photocatalytic lamp tube is matched with the structure of electric fan. Ultraviolet (UV) light emitted by a UV lamp tube with titanium dioxide catalytic materials sleeved outside is exploited to perform sterilization and cleaning of air. Most harmful gases detrimental to human body will thus be decomposed into harmless gases because of photocatalytic reaction letting the gases generate hydrogen and oxygen radicals, thereby achieving purification of air. Therefore, air can conform to environmental requirement, and quality of air around human body can be enhanced.

To achieve the above object, a photocatalytic electric fan of the present invention comprises a main body having a receiving tank therein. A motor is fixed in the receiving tank. A rotation shaft of the motor is connected with a fan blade. A back net, being a reticular lamp-fixing element, is joined at an opening of the main body near the side of the fan blade. A photocatalytic lamp is positioned on the back net. At least a photocatalytic lamp tube is disposed on the lamp. Each photocatalytic lamp tube is connected to an electric connection seat, a stabilizer, a starter, a switch, and a power source. A face net is joined at the other opening of the main body. The face net is rotatable. If the face net is not rotatable, it becomes another reticular lamp-fixing element, i.e., a photocatalytic lamp is also positioned on the inner face of the face net. The photocatalytic lamp tube is a UV lamp tube with a glass-fiber-cloth sleeved outside. The glass-fiber-cloth has a photocatalytic coating, which is formed of titanium dioxide. The reticular lamp-fixing element can also be a plurality of holder rods in the main body. Inner ends of the holder rods are joined with a central lump having a receiving tank.

The various objects and advantages of the present invention will be more readily understood from the following detailed description when read in conjunction with the appended drawing, in which:

### BRIEF DESCRIPTION OF THE DRAWINGS

FIG. 1 is an exploded perspective view of a conventional electric fan;

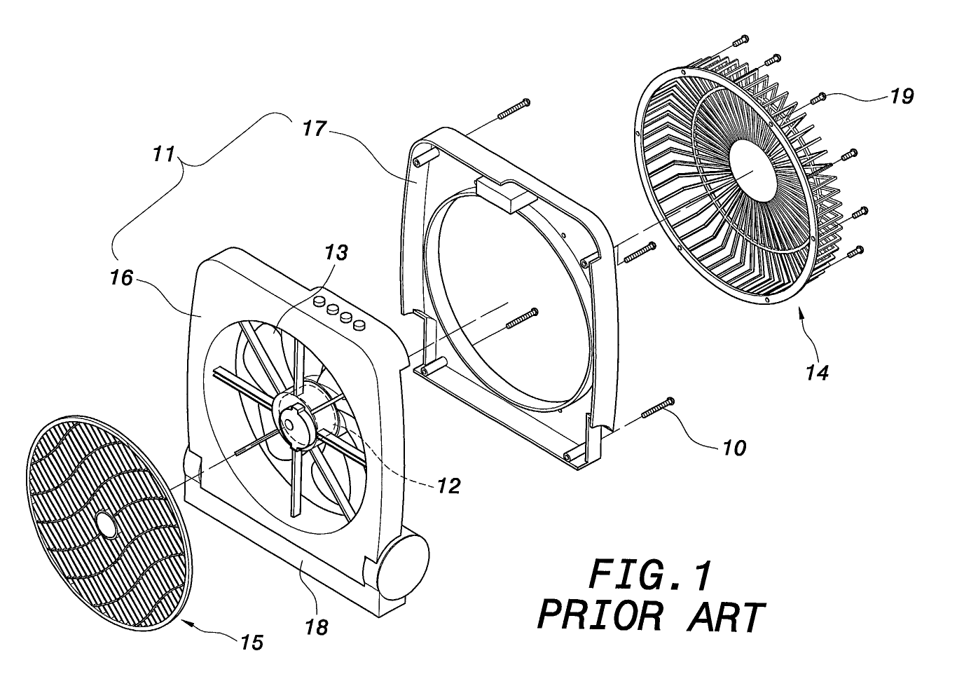

FIG. 2 is a perspective view of the present invention embodied on a box type electric fan;

FIG. 3 is an exploded perspective view of FIG. 2;

FIG. 4 is an exploded perspective view of the present invention with the lamp disposed at holder rods of the main body;

FIG. 5 is an exploded perspective view of the present invention with a dual-ring type lamp used;

FIG. 6 is an exploded perspective view of the present invention with a pair of straight-tube type lamps used;

FIG. 7 is a perspective view of the present invention embodied on a stand-type electric fan;

FIG. 8A shows a decomposition curve of gas of ethyl acetate of the present invention (22W/40W);

FIG. 8B shows a decomposition curve of gas of ethyl acetate of the present invention (22W/32W);

FIG. 9A shows a decomposition curve of gas of acetic acid of the present invention (22W/32W); and

FIG. 9B shows a decomposition curve of gas of acetic acid of the present invention (22W/40W).

### DETAILED DESCRIPTION OF THE PREFERRED EMBODIMENTS

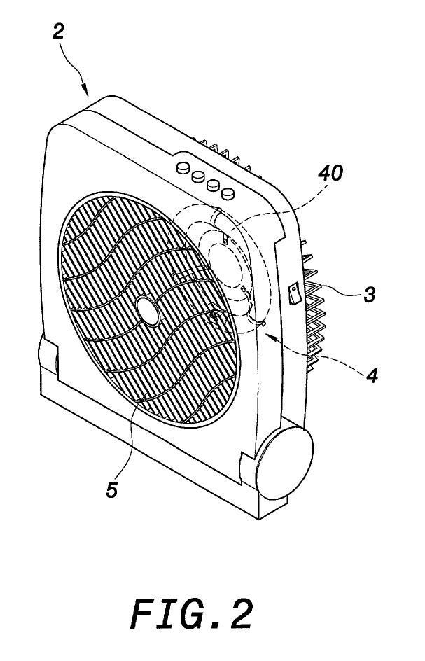

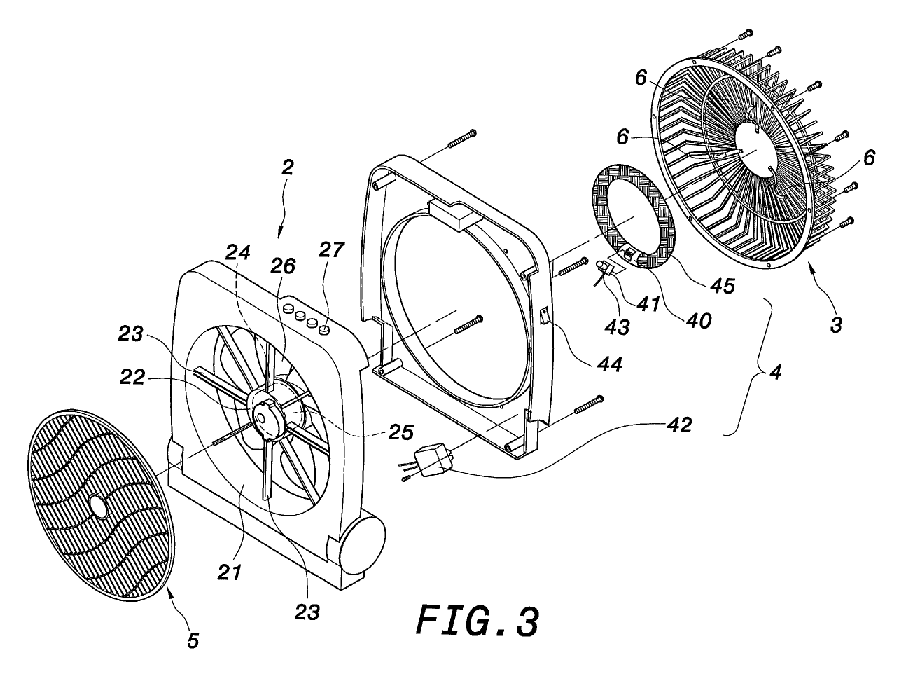

As shown in FIGS. 2 and 3, a photocatalytic electric fan of the present invention comprises a main body 2. The electric fan shown in the figure is a box type electric fan. Two sides of the main body 2 are open. That is, a transversal through circular hole 21 is disposed in the main body 2. Openings are formed at two end faces of the circular hole 21. A central lump 22 is disposed on the main body 2. A plurality of radial holder rods 23 are disposed on the inner peripheral face of the circular hole to connect the central lump 22. A receiving tank 24 is formed in the central lump 22 with a motor 24 fixedly placed therein. The rotation shaft of the motor 25 is pivotally joined with a fan blade 26. A back net 3 is joined at the opening of the main body 2 near the fan blade 26. The back net is a reticular lamp-fixing element.

A photocatalytic lamp 4 is positioned on the inner face of the back net 3. At least a photocatalytic lamp tube 40 is disposed on the lamp 4. Each photocatalytic lamp tube 40 is connected with an electric connection seat 41, a stabilizer 42, a starter 43, a switch 44, and a power source (not shown). The power source is connected to the switch 44 with electric wires so that the power source can be an independent power source, i.e., having an independent plug (not shown), or the power source of the lamp 4 is directly connected in series with the power source of the motor 25, or the switch 44 is connected with a switch 27 of the electric fan, or the switch 44 is joined on the main body 2 (as shown in the figure). The switch 44 is used to independently open or close the lamp 4, or can synchronously open or close the lamp 4 and the motor 25 of the electric fan. The photocatalytic lamp tube 40 is an UV lamp tube. A glass-fiber-cloth 45 is sleeved outside the lamp tube 40. The glass-fiber-cloth 45 has a photocatalytic coating formed of titanium dioxide.

A face net 5 is joined at the other opening of the main body 2 away from the fan blade 26. The face net 5 is rotatable. The face net 5 is led to move by the other end of the motor 25. This is a detailed structure of electric fan and thus will not be further illustrated. If the face net 5 is not rotatable, it becomes another reticular lamp-fixing element. A photocatalytic lamp 4 can be positioned on the inner or outer face of the face net 5. The portion where the lamp 4 is disposed is not shown in the figure, but can be directly deduced from the embodiment of FIG. 3.

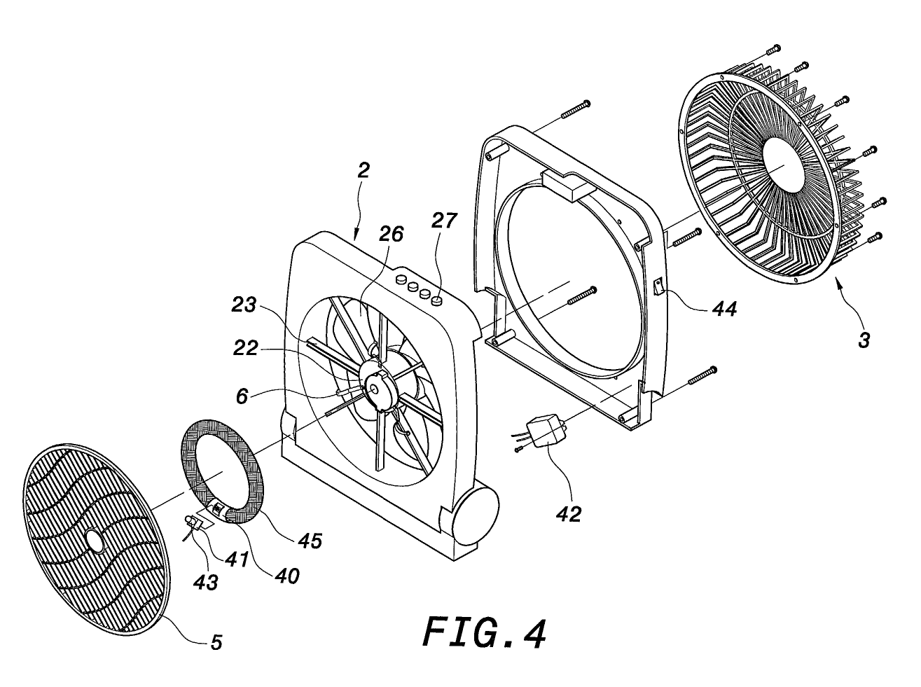

Additionally, another type of the reticular lamp-fixing element is the position of the holder rods 23 or the central lump 22, as shown in FIG. 4. Similarly, at least a lamp-positioning element 6 is disposed at a predetermined position of the central lump 22. The lamp-positioning element 6 is a panel of slightly curved shape. One end of the lamp-positioning element 6 is fixed on the central lump 22 of the reticular lamp-fixing element, and the other end thereof is hooked with the lamp tube 40 of the lamp 4 or at the electric connection seat 41.

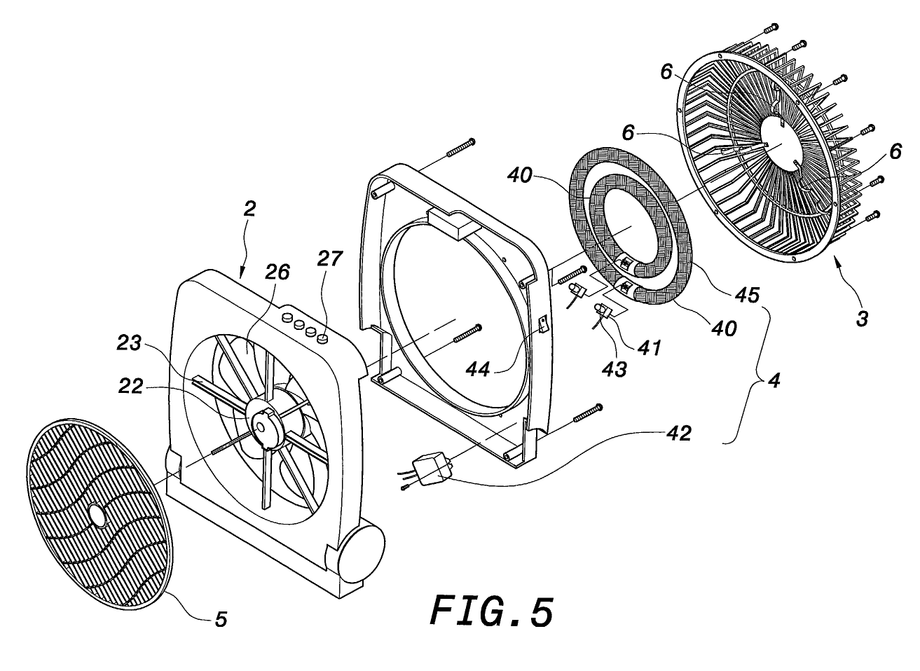

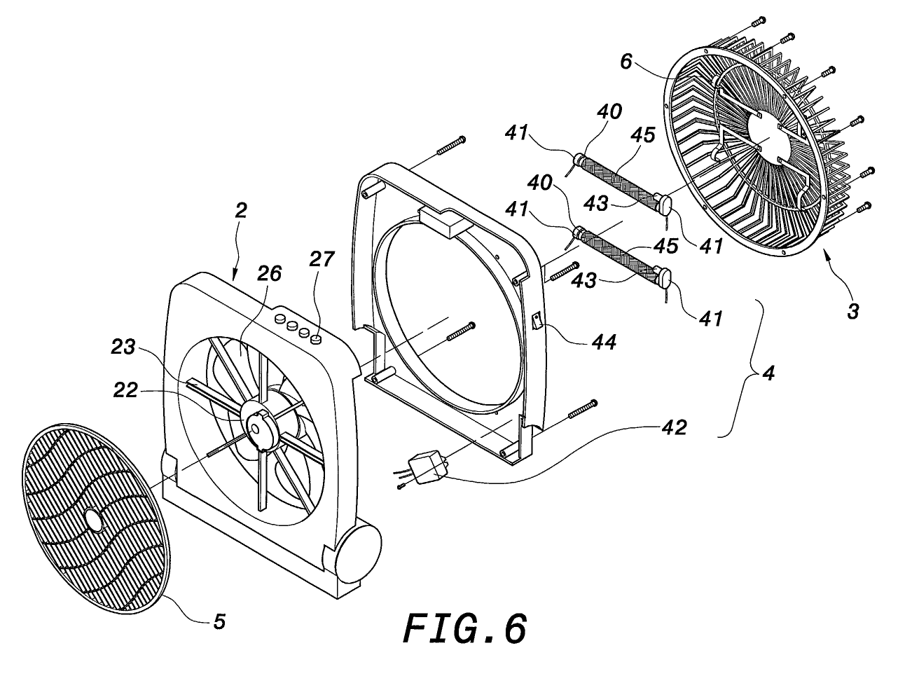

As shown in FIG. 5, the lamp tube 40 is changed from a single-ring type shown in FIG. 3 to a dual-ring type lamp. As shown in FIG. 6, the lamp tube 40 is changed from a single-ring type shown in FIG. 3 to a pair of straight-tube type lamps. The number of straight-tube lamp shown in FIG. 6 can increase or decrease according to necessity. The number of dual-ring lamp shown in FIG. 5 can also increase. The lamp tubes should be disposed in pair with the electric connection seat, the starter, and the stabilizer.

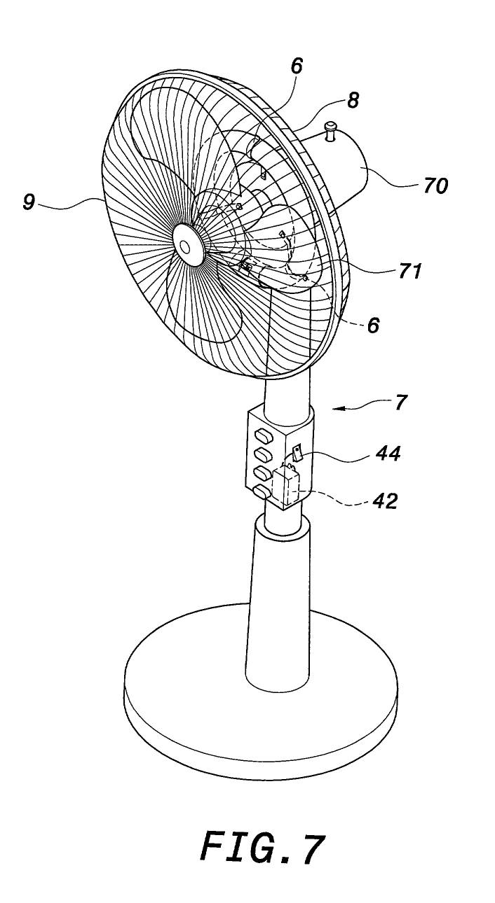

As shown in FIG. 7, a conventional stand-type electric fan capable of turning left and right comprises a seat body 7. The upper end of the seat body 7 is a main body 70 with a receiving tank (not shown) disposed therein. A motor (not shown) is fixedly placed in the receiving tank. The rotation shaft of the motor is joined with a fan blade 71. A back net 8 and a face net 9 cover outside the fan blade 71 of the main body 7. The back net 8 and the face net 9 are reticular lamp-fixing elements. That is, the lamp 4 is disposed on the back net 8 or the face net 9, or the lamps 4 are disposed on both the back net 8 and the face net 9. In this figure, the lamp 4 is disposed only on the back net 8. A plurality of lamp-positioning elements 6 are disposed on the inner face of the back net 8. The lamp-positioning elements 6 are used to position at least a lamp tube 40, which is a ring-type lamp tube here. The lamp tube 40 is connected to an electric connection seat (not shown). The electric connection seat is connected with a stabilizer 42, a starter (not shown), and a switch 44. The switch 44 is joined on the seat body 7. The main body said in the claim also comprises the seat body 6 in this embodiment. The stabilizer 7 is disposed in the seat body 7 in this figure to form another embodiment of the present invention.

The lamp tube 40 is a UV lamp tube emitting UV light of wavelength 200˜750 nm, and preferably 300˜400 nm. The lamp tube capable of cleaning air is a lamp tube sleeved by a glass-fiber-cloth, which has a photocatalytic coating. Please refer to “UV lamp for air cleaning” disclosed in R.O.C. Pat. No. 87,113,380 and U.S. Pat. No. 6,135,838 for the disposition of the photocatalytic coating and the glass-fiber-cloth, wherein the method of manufacturing the lamp tube is described in detail. Because the lamp tube is not for illumination, it can match the specified light of appropriate wavelength and the materials coated on the lamp tube to treat exhaust gases.

The lamp tube emits UV light to achieve the function of sterilization. The lamp tube can decomposes exhaust gases near it. In other words, decomposition reaction caused by the glass-fiber-cloth and metallic photocatalytic coating of titanium dioxide is exploited to further clean air for reducing the amount of exhaust gases. Titanium dioxide of the photocatalytic coating is illuminated by UV light and excited by accepting photons to generate free electron-hole pairs, letting electrons easily combine with oxygen to form organic oxygen and holes easily combine with hydroxyl to form free radicals of hydroxyl. Meanwhile, oxygen on the surface of the photocatalytic coating generates unstable ions having oxidizing capability, which will generate chemical combination and decomposition reaction when contacting organic or inorganic gases in air. Under the condition of continual supply, harmful exhaust gases in air can be decomposed into harmless gases such as dioxide and water. When the reaction process is initially started, free radicals of hydroxyl are generated to cause relevant reactions with various kinds of organic and inorganic pollution sources, altering the characteristics of the pollution sources to become in a stable state.

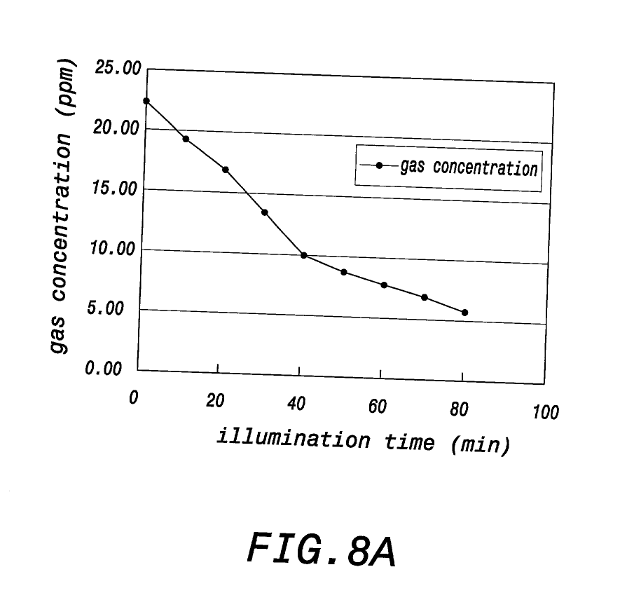

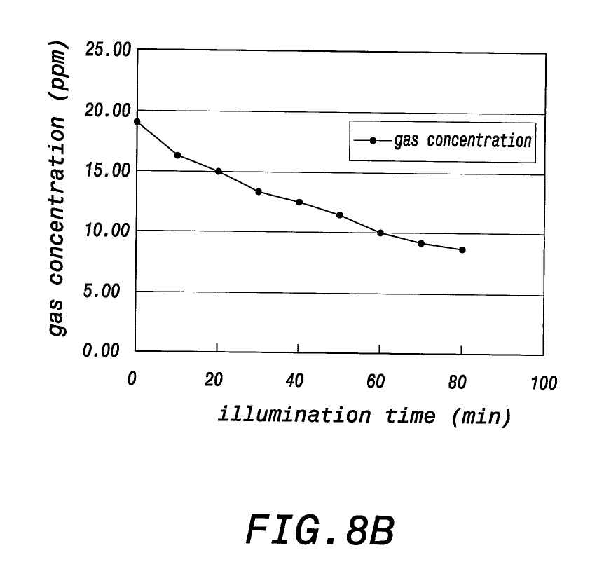

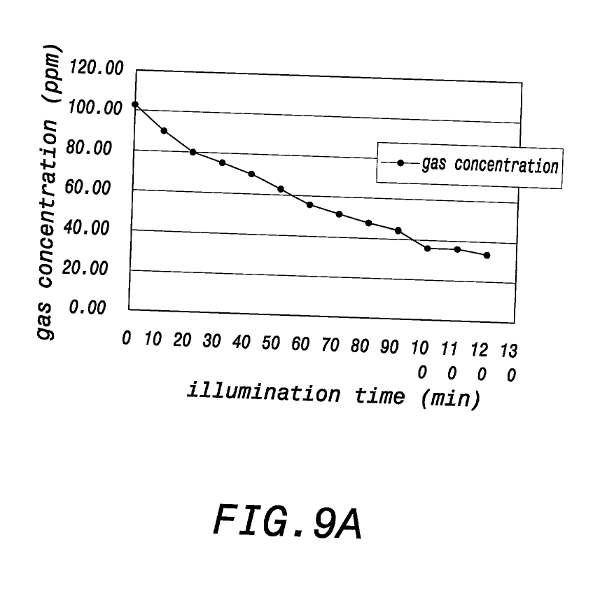

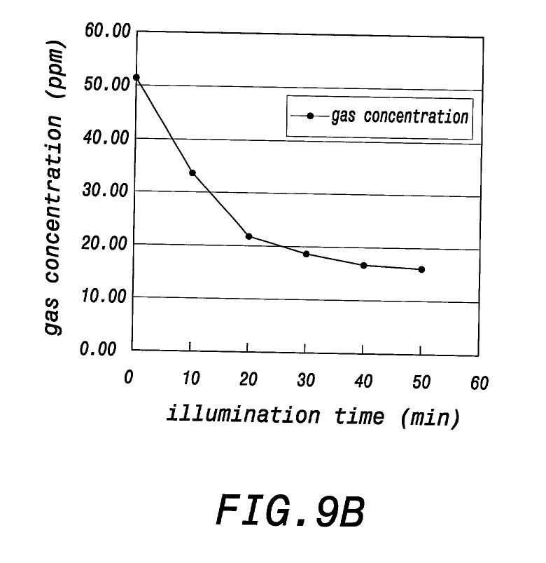

The present invention utilizes the above specially designed structure formed by joining a photocatalytic lamp tube for sterilization and cleaning of air and an electric fan, hence achieving the function of decomposing exhaust gases to obtain the functions of sterilization, suppressing growth of bacterium, and eliminating malodor and bad and mixed smell. Moreover, exhaust gases can be decomposed to generate hydroxyl to further clean air, thereby conforming to environmental requirement and letting environmental quality be better. As shown in FIGS. 8 and 9, the present invention can operate under the conditions of 22W/32W and 22W/40W to reduce the concentration of exhaust gases after a period of time. The present invention can also be used for waste gases such as acetic acid, tetrahydrocannabinol, ethyl methyl ketone, n-Hexane, methyl alcohol, butyl acetate, pyridine, methane dichloride, trimethylamine, methanal, methylbenzene, and acrylonitrile. Very good reaction speed of oxidation and decomposition can be obtained. The present invention can thus provide very good usage.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A photocatalytic electric fan, comprising:
a main body having a receiving tank;
a motor fixedly placed in said receiving tank and having a rotation shaft;
a fan blade fixed on said rotation shaft of said motor;
a reticular lamp-fixing element fixedly joined on said main body; and
a lamp being a photocatalytic lamp, said lamp being positioned by said reticular lamp-fixing element, said lamp having at least a photocatalytic lamp tube, a glass-fiber-cloth being sleeved outside said photocatalytic lamp tube, said glass-fiber-cloth having a photocatalytic coating formed of titanium dioxide, each said lamp tube being connected to an electric connection seat, said electric connection seat being connected to a stabilizer, a starter, and a power source, said starter and said starter being disposed in said main body.

2. The photocatalytic electric fan as claimed in claim 1, wherein said reticular lamp-fixing element is a back net and a face net covering said fan blade, and lamps are disposed on said back net or said face net or both.

3. The photocatalytic electric fan as claimed in claim 1, wherein the wavelength of light emitted by said photocatalytic lamp tube is between 200˜760 nm.

4. The photocatalytic electric fan as claimed in claim 1, wherein said photocatalytic lamp tube is an ultraviolet lamp tube.

5. The photocatalytic electric fan as claimed in claim 1, wherein said photocatalytic lamp tube is of straight-tube shape or ring-tube shape.

6. The photocatalytic electric fan as claimed in claim 1, wherein a pair of said photocatalytic lamp tubes are provided.

7. The photocatalytic electric fan as claimed in claim 1, wherein two sides of said main body are open, and a plurality of holder rods are disposed in said main body to connect a central lump, said receiving tank being disposed in said central lump, a back net being joined at an opening of said main body near the side of said fan blade, said reticular lamp-fixing element being said back net and/or the position of said central lump and said holder rods, said reticular lamp-fixing element being used to connect said lamp.

8. The photocatalytic electric fan as claimed in claim 7, wherein a face net is disposed at the other opening of said main body, and said face net is rotatable.

9. The photocatalytic electric fan as claimed in claim 7, wherein a face net is disposed at the other opening of said main body, and said reticular lamp-fixing element is said face net, said reticular lamp-fixing element being used to connect said lamp.

10. The photocatalytic electric fan as claimed in claim 1, wherein said power source of said lamp is connected with a power source of said motor.

11. The photocatalytic electric fan as claimed in claim 1, wherein a switch is disposed on said power source of said lamp, and said switch is joined on said main body.

12. The photocatalytic electric fan as claimed in claim 1, wherein a plug is disposed on said power source of said lamp.

13. The photocatalytic electric fan as claimed in claim 1, wherein said reticular lamp-fixing element has at least a lamp-positioning element, said lamp-positioning element being a panel of slightly curved shape, one end of said lamp-positioning element being fixed on said reticular lamp-fixing element, the other end thereof being hooked with said lamp tube of said lamp or at said electric connection seat.
